(19) United States
(12) Patent Application Publication
(10) Pub. No.: US 2011/0282224 A1
(43) Pub. Date: Nov. 17, 2011

(54) PULSE WAVE ANALYZER AND PULSE WAVE ANALYZING METHOD

(75) Inventors: Hironori Sato, Moriyama-shi (JP); Tatsuya Kobayashi, Otsu-shi (JP); Hideaki Yoshida, Kyoto-shi (JP); Kenji Fujii, Kyoto-shi (JP); Toshihiko Ogura, Inuyama-shi (JP)

(73) Assignee: OMRON HEALTHCARE CO., LTD., Kyoto (JP)

(21) Appl. No.: 13/189,634

(22) Filed: Jul. 25, 2011

Related U.S. Application Data
(63) Continuation of application No. PCT/JP2010/051118, filed on Jan. 28, 2010.

(30) Foreign Application Priority Data
Feb. 3, 2009 (JP) 2009-022972

(51) Int. Cl. A61B 5/02

(52) U.S. Cl. 600/500

(57) ABSTRACT

In a pulse wave analyzer, a local maximum point of a fourth order differentiated wave of the pulse wave of one beat is acquired, and a maximum point of a reflection wave of the local maximum points of the fourth order differentiation existing in a zone of an original waveform is determined as a starting point of a reflection wave zone that is a first characteristic point. With 10% of the amplitude of the first characteristic as a threshold value, a time point at which the amplitude reaches the threshold value after the relevant point is determined as an ending point of the reflection wave zone that is a second characteristic point. The duration time of the reflection time of the time between the first characteristic point and the second characteristic point is calculated as an index useful in the diagnosis of heart disease.

### TECHNICAL FIELD

The present invention relates to pulse wave analyzers and pulse wave analyzing methods, and in particular, to a pulse wave analyzer and a pulse wave analyzing method for calculating a characteristic point of a pulse wave.

### BACKGROUND ART

One of the information useful in diagnosing cardiovascular disease such as arterial sclerosis is the transmission timing or the occupying time of the reflection wave in the pulse wave. In order to obtain the time where the reflection wave in the pulse wave exists, an analysis for dividing the measured pulse wave to the range of ejection wave and the range of reflection wave is required.

In Japanese Unexamined Patent Publication No. 2005-349116 (hereinafter referred to as patent document 1), the applicant of the present application proposes a pulse analyzer for extracting a characteristic point of a pulse wave, and calculating an index such as an Al (Augmentation Index) or a TR (Traveling time to Reflected wave). The index such as the Al and the TR is an index that is calculated by extracting the rising point of the synthetic wave of the rising point of the reflection wave as the characteristic point.

In the document Increased Systolic Pressure in Chronic Uremia Role of Arterial Wave Reflections, London et al proposes a method of analyzing the characteristics of the pulse wave obtained only from one point on the artery and obtaining the index such as the TR by extracting the wave reflected from the branched portion of the iliac artery.
Patent Document 1: Japanese Unexamined Patent Publication No. 2005-349116
Non-Patent Document 1: London et al. “Increased Systolic Pressure in Chronic Uremia Role of Arterial Wave Reflections,” Hypertension, vol. 20, No. 1, 1992, pp. 10-19

### SUMMARY OF INVENTION

However, the rising point of the reflection point is difficult to be accurately extracted from the synthetic wave, and in particular, the rising point of the reflection wave may be hard to appear in the synthetic wave depending on the measuring site. If the rising point of the reflection wave is not extracted, the index cannot be calculated with the method disclosed in document 1. Non-patent document 1 relates to a technique of capturing a different phenomenon and calculating the index, but the technique is difficult to apply to the pulse wave measured at the upper arm that can be measured at home.

Therefore, one or more embodiments of the present invention provides a pulse wave analyzer and a pulse wave analyzing method capable of extracting a convergence time of the reflection wave and calculating the index useful in the diagnosis of the heart disease.

According to one or more embodiments of the present invention, a pulse wave analyzer includes a pulse wave detection unit for detecting a pulse wave; and a calculation device for carrying out a process based on the pulse wave detected by the pulse wave detection unit; wherein the process carried out by the calculation device includes a process of extracting a characteristic point for sectionalizing a reflection wave zone from a pulse wave waveform of one beat, and a process of calculating a convergence time of the reflection wave as an index.

According to one or more embodiments of the present invention, a pulse wave analyzing method includes the steps of extracting a characteristic point for sectionalizing a reflection wave zone from a pulse wave waveform of one beat obtained with a pressure sensor for detecting a pulse wave; and calculating a convergence time of a reflection wave as an index.

According to one or more embodiments of the present invention, a pulse wave analyzing program is a program for causing a computer to execute a process of analyzing a pulse wave and calculating an index; the program causing the computer to execute the steps of acquiring a sensor signal from a pressure sensor for detecting a pulse wave; extracting a characteristic point for sectionalizing a reflection wave zone from a pulse wave waveform of one beat based on the sensor signal; and calculating a convergence time of a reflection wave as an index.

According to one or more embodiments of the present invention, the convergence time of the reflection wave can be extracted. The pulse wave can be automatically analyzed even when the rising point of the reflection wave is not extracted by using such index.

### BRIEF DESCRIPTION OF THE DRAWINGS

FIG. 1 is a view showing a specific example of a device configuration of a pulse wave analyzer according to one or more embodiments of the present invention.

FIG. 2 is a view showing a relationship of a pulse wave propagation time (PTT: Pulse Transmission Time) and a duration time (TRD: Traveling time of Reflection wave Duration) of the reflection wave in the measuring pulse wave between a forearm and an ankle.

FIG. 3 is a view showing a relationship of the PTT and the TRD between a neck and a femoral area.

FIG. 4 is a view showing a relationship of a propagation speed (PWV: Pulse Wave Velocity) of the pulse wave and the TRD between the forearm and the ankle.

FIG. 5 is a view showing a relationship of the PWV and the TRD between the neck and the femoral area.

FIG. 6 is a flowchart showing an analyzing process of a pressure signal (sensor signal) obtained from a sensor element of a semiconductor pressure sensor 19 in the pulse wave analyzer according to one or more embodiments of the present invention.

FIG. 7 is a view showing a specific example of a relationship between a pulse wave waveform, a primary differentiated wave and a secondary differentiated wave.

FIG. 8A is a view showing the characteristics of a zero crossing point.

FIG. 8B is a view showing the characteristics of the zero crossing point.

FIG. 8C is a view showing the characteristics of the zero crossing point.

FIG. 9 is a view showing a usage example of a fourth order differentiation.

FIG. 10 is a view describing the frequency characteristics of the fourth order differentiation filter.

FIG. 11 is a flowchart showing a specific flow of the process of extracting a characteristic point in the pulse wave analyzer according to one or more embodiments of the present invention.

FIG. 12 is a view showing a specific example of a band pass filter used in the pulse wave analyzer according to one or more embodiments of the present invention.

### DETAILED DESCRIPTION OF INVENTION

Embodiments of the present invention will be hereinafter described with reference to the drawings. In the following description, the same reference numerals are denoted for the same components and configuring elements. The names and functions thereof are also the same.

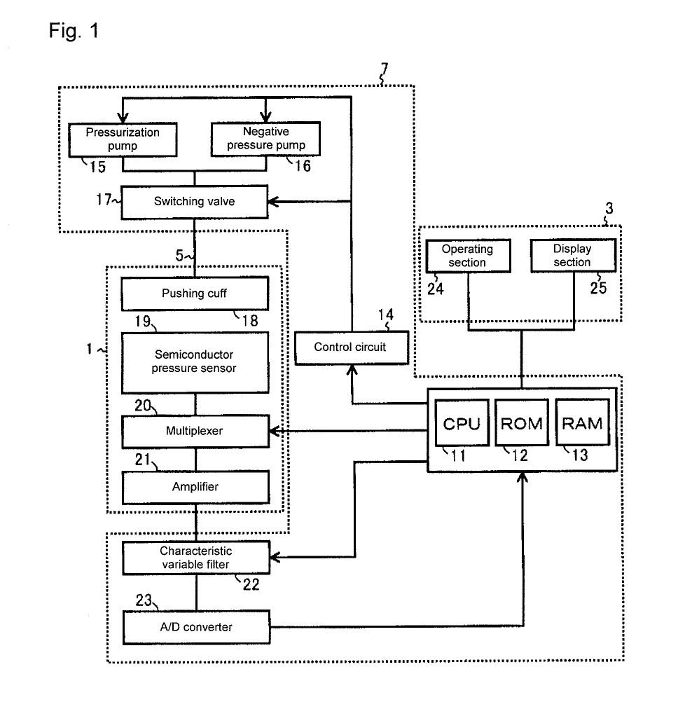

With reference to FIG. 1, a pulse wave analyzer according to one or more embodiments of the present invention includes a sensor unit 1, a display unit 3, and a fixing stand unit 7.

The display unit 3 includes an operating section 24 arranged to be operable from the outside so as to be operated to input various types of information related to pulse wave analysis or the like, and a display section 25 including an LED (Light Emitting Diode) or an LCD (Liquid Crystal Display) for outputting various types of information such as the pulse wave analysis result to the outside.

The fixing stand unit 7 includes a ROM (Read Only Memory) 12 and a RAM (Random Access Memory) 13 for storing data and programs for controlling the pulse wave analyzer, a CPU (Central Processing Unit) 11 for executing various processes including calculation to intensively control the pulse wave analyzer, a pressurization pump 15, a negative pressure pump 16, a switching valve 17, a control circuit 14 for receiving a signal from the CPU 11 and transmitting to the pressurization pump 15, the negative pressure pump 16, and the switching valve 17, a characteristic variable filter 22 that can be changed to at least two values, and an A/D converter 23.

The CPU 11 accesses the ROM 12 and reads out the program, and develops and executes the program on the RAM 13 to control the entire pulse wave analyzer. The CPU 11 receives an operation signal from the user by the operating section 24, and controls the entire pulse wave analyzer based on the operation signal. In other words, the CPU 11 transmits the control signal to the control circuit 14, a multiplexer 20, and the characteristic variable filter 22 based on the operation signal input from the operating section 24. The CPU 11 also performs a control to display the pulse wave analysis result, or the like on the display section 25.

The pressurization pump 15 is a pump for pressurizing the inner pressure (hereinafter referred to as “cuff pressure”) of the pushing cuff (air bladder) 18 to be described later, and the negative pressure pump 16 is a pump for depressurizing the cuff pressure. The switching valve 17 selectively switches and connects either the pressurization pump 15 or the negative pressure pump 16 to the air tube 5. The control circuit 14 controls them according to a control signal from the CPU 11.

The sensor unit 1 includes a semiconductor pressure sensor 19 including a plurality of sensor elements, a multiplexer 20 for selectively deriving a pressure signal output by each of the plurality of sensor elements, an amplifier 21 for amplifying the pressure signal output from the multiplexer 20, and a pushing cuff 18 including an air bladder pressure-adjusted to push the semiconductor pressure sensor 19 on the measurement site.

The semiconductor pressure sensor 19 includes a plurality of sensor elements arrayed at a predetermined interval in one direction on a semiconductor chip made of monocrystal silicon, and is pushed against the measurement site being measured such as the upper arm by the pressure of the pushing cuff 18. The semiconductor pressure sensor 19 detects the pulse wave of the subject through the radial artery in such state. The semiconductor pressure sensor 19 inputs the pressure signal output by detecting the pulse wave to the multiplexer 20 for every channel of each sensor element. Forty sensor elements are arrayed by way of example.

The multiplexer 20 selectively outputs the pressure signal output by each sensor element. The pressure signal provided from the multiplexer 20 is amplified by the amplifier 21, and selectively output to the A/D converter 23 through the characteristic variable filter 22.

According to one or more embodiments of the present invention, the multiplexer 20 sequentially switches the plurality of pressure signals output from the plurality of sensor elements and outputs the same according to the control signal from the CPU 11 until an optimum sensor element for pulse wave detection is selected. The channel is fixed according to the control signal from the CPU 11 after the optimum sensor element for pulse wave detection is selected. In this case, the multiplexer 20 selects and outputs the pressure signal output from the selected sensor element.

The characteristic variable filter 22 is a low pass filter for cutting off the signal component of greater than or equal to a predetermined value, and can be changed to at least two values.

The A/D converter 23 converts the pressure signal, which is an analog signal, derived from the semiconductor pressure sensor 19 to digital information, and provides the same to the CPU 11. The pressure signal output by each sensor element included in the semiconductor pressure sensor 19 is simultaneously acquired through the multiplexer 20 until the channel of the multiplexer 20 is fixed by the CPU 11. After the channel of the multiplexer 20 is fixed by the CPU 11, the pressure signal output from the relevant sensor element is acquired. The period at which the pressure signal is sampled (hereinafter referred to as “sampling period”) is, for example, 2 ms.

The characteristic variable filter 22 described above changes the value of the cutoff frequency for until the channel of the multiplexer 20 is fixed and for after the channel is fixed. The sampling is carried out while switching the plurality of pressure signals until the channel of the multiplexer 20 is fixed. Therefore, the value of the cutoff frequency higher than the sampling frequency (e.g., 20 kHz) in this case is selected. The undulation thus can be prevented from occurring after the A/D conversion, and an optimum sensor element can be appropriately selected. After the channel is fixed, the value that becomes the cutoff frequency of smaller than or equal to ½ of the sampling frequency (e.g., 500 Hz) with respect to one certain pressure signal is selected according to the control signal from the CPU 11. The aliasing noise thus can be removed, and the pulse wave analysis can be accurately carried out. The aliasing noise refers to a noise having a frequency component of greater than or equal to ½ of the sampling frequency that appears in the region of smaller than or equal to ½ of the sampling frequency by the turnover phenomenon when converting the analog signal to the digital signal by a sampling theorem.

According to one or more embodiments of the present invention, the display unit 3 can be miniaturized because the CPU 11, the ROM 12, and the RAM 13 are arranged in the fixing stand unit 7.

The fixing stand unit 7 and the display unit 3 are separately arranged, but the display unit 3 may be incorporated in the fixing stand unit 7. On the contrary, the CPU 11, the ROM 12, and the RAM 13 may be arranged in the display unit 3. The PC (Personal Computer) may be connected to carry out various types of controls.

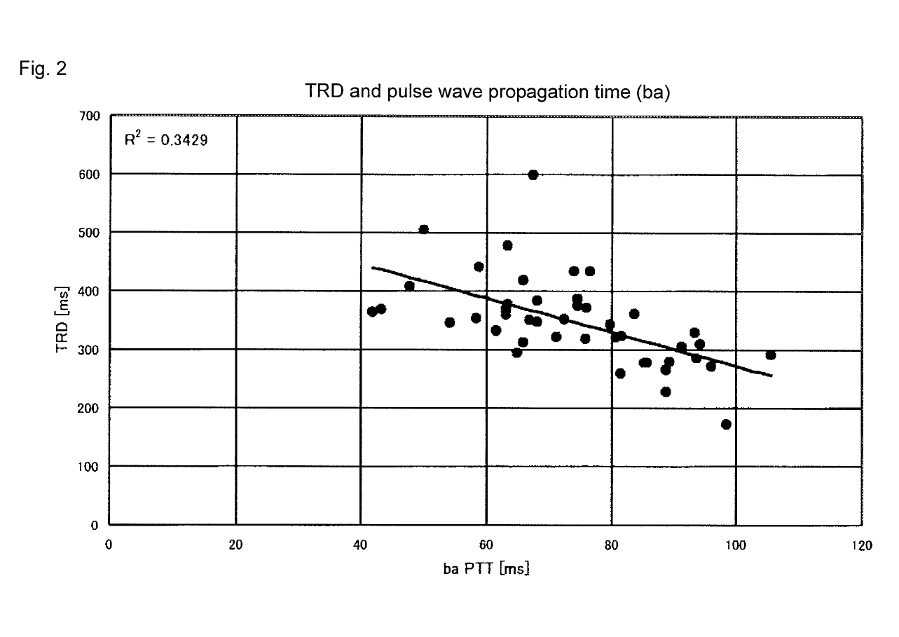

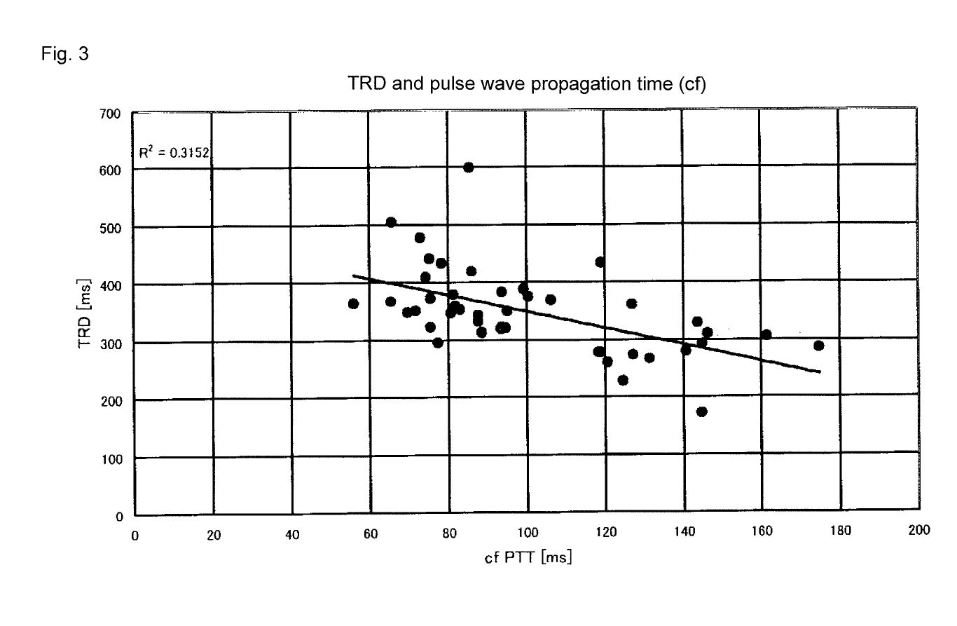

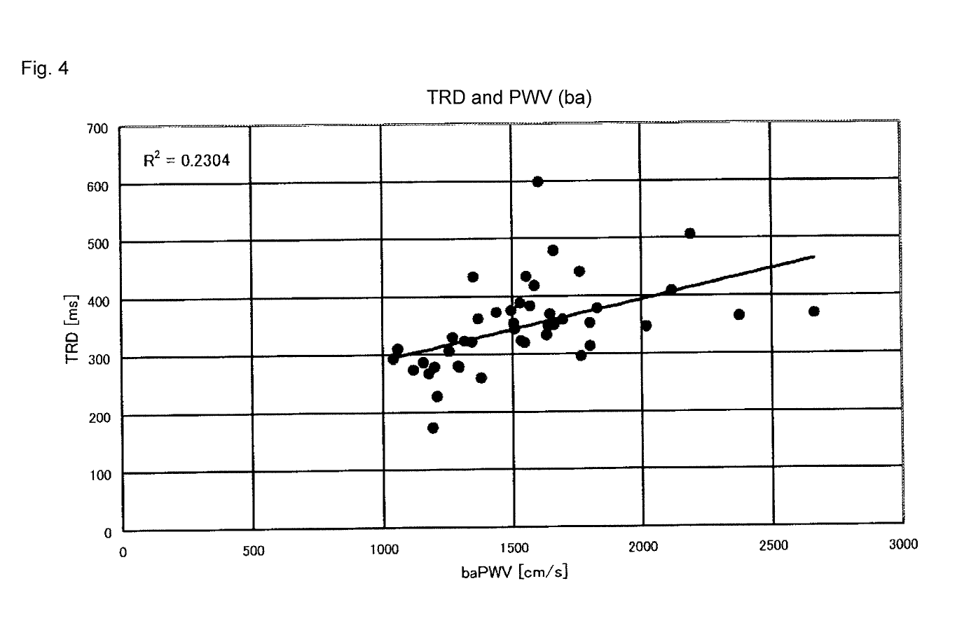

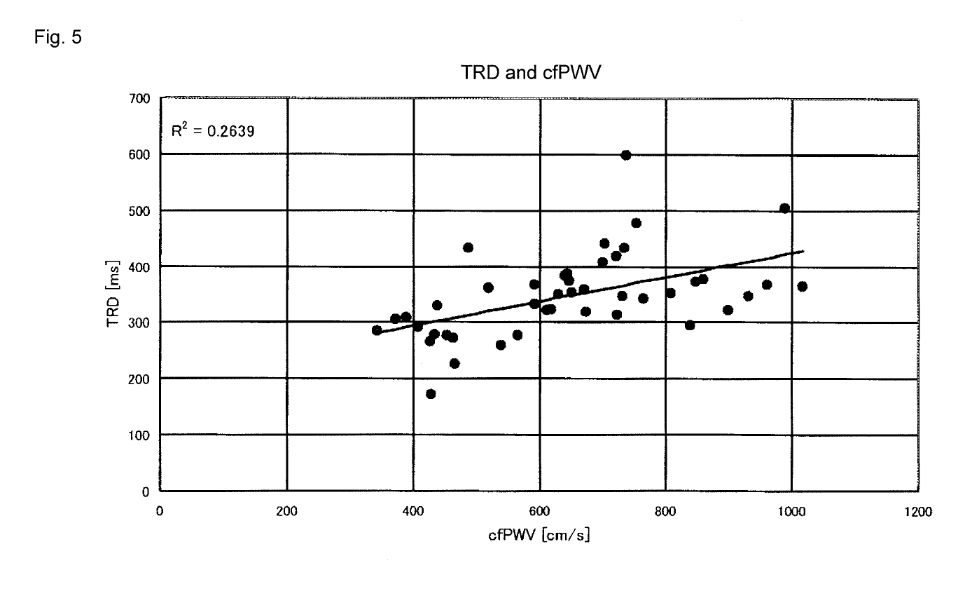

According to one or more embodiments of the present invention, the pulse wave analyzer calculates the duration time of the reflection wave in the measuring pulse wave (hereinafter referred to as TRD: Traveling time of Reflection wave Duration) as an index useful for diagnosing heart disease such as arterial sclerosis from the pulse wave waveform. Because the propagating speed of the pulse wave ejected from the heart becomes faster as the arterial sclerosis advances, the propagation speed of the pulse wave (hereinafter referred to as PWV: Pulse Wave Velocity) is assumed as an effective index in diagnosing heart diseases such as arterial sclerosis. There is a correlation between the pulse wave propagation time (hereinafter referred to as PTT: Pulse Transmission Time) and the TRD, which may be calculated from a great number of pulse wave samples. FIG. 2 shows the relationship of the PTT and the TRD between a forearm and an ankle, and FIG. 3 shows the relationship of the PTT and the TRD between a neck and a femoral area. Similarly, there is a correlation between the PWV and the TRD, which may be calculated from a great number of pulse wave samples. FIG. 4 shows the relationship of the PWV and the TRD between the forearm and the ankle, and FIG. 5 shows the relationship of the PWV and the TRD between the neck and the femoral area. According to such verification, the TRD can also be an effective index in diagnosing heart diseases such as the arterial sclerosis.

The measured pulse wave needs to be separated to a reflection wave existing zone and a reflection wave non-existing zone in order to calculate the TRD from the measured pulse wave. The former zone of the two zones is a zone in which the vibration is extracted because the high frequency component exists in the measured pulse wave for one beat that is the synthetic wave, and the latter zone is a zone in which the vibration is not extracted because the high frequency component does not exist. In other words, the former zone can be referred to as a vibration zone and the latter zone can be referred to as a stable zone. The pulse wave analyzer according to one or more embodiments of the present invention extracts a starting point and an ending point of at least one zone of the two zones as characteristic points from the measured pulse wave to extract the two zones.

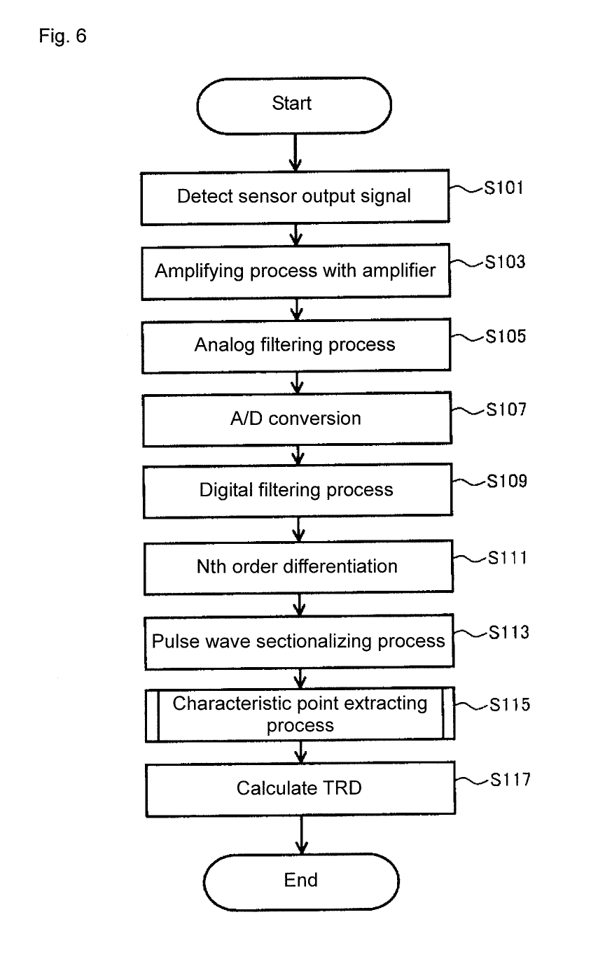

The process shown in the flowchart of FIG. 6 is realized when the CPU 11 in the fixing stand unit 7 accesses the ROM 12 to read out the program, and develops and executes the same on the RAM 13. At least a part of the process may be realized by hardware configuration shown in FIG. 1. This process will be described as an analyzing process after the channel of the multiplexer 20 is fixed.

With reference to FIG. 6, when detecting the pressure signal in step S101, the semiconductor pressure sensor 19 including a plurality of sensor elements inputs the pressure signal to the multiplexer 20. In this case, the sensor signal output from the sensor element corresponding to the fixed channel is selected by the multiplexer 20. The pressure signal selected by the multiplexer 20 is input to the amplifier 21.

The amplifier 21 amplifies the pressure signal to a predetermined amplitude in step S103, and the characteristic variable filter 22 performs an analog filtering process in step S105. In this case, the characteristic variable filter 22 cuts off the signal component of smaller than or equal to ½ of the sampling frequency. If the sampling frequency is 500 Hz, the signal component having a frequency exceeding 100 Hz is cut off.

The A/D converter 23 digitizes the pressure signal passed through the characteristic variable filter 22 in step S107, and executes a digital filtering process for extracting a frequency of a predetermined range in an aim of removing noise, or the like in step S109. The A/D converter 23 transfers the digitized pressure signal to the CPU 11.

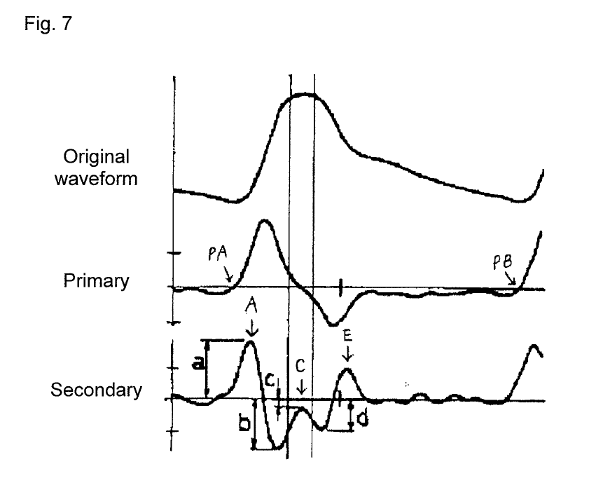

In step S111, the CPU 111 receives the pressure signal from the ND converter 23 and takes a difference of each data to perform differentiation of first to fifth order. The CPU 11 performs N^{th} order differentiation on the pulse wave waveform obtained from the pressure signal by executing the program stored in the ROM 12. In step S113, the CPU 11 sectionalizes the pulse wave waveform based on the differentiation result and extracts the pulse wave waveform for one beat. Specifically, the CPU 11 waits until the first differentiation of the Nth order differentiation acquired in step S111 becomes positive. When the first differentiation becomes positive, a rising zero crossing point thereof is maintained and set as a “temporary rising point”. The CPU 11 then waits for a local maximum value of the first differentiation. When detecting the local maximum of the first differentiation, the CPU 11 determines whether one beat is recognized. Specifically, with reference to FIG. 7, when the CPU 11 waits for the local maximum value of the original waveform and detects the local maximum value, the CPU 11 references the waveform from a temporary rising point (PA point) immediately before to a rising point (PB point) before that. A maximum point (PP point) of the original waveform is confirmed to exist between the PA point and the PB point, and the PB point is confirmed to be a minimum value between the PP point and the PB point. If confirmed that the PB point is a minimum value, the PA point is set as a “rising point”. The pulse wave waveform of one beat then becomes from the PA point to the PB point. The PA point can also be defined as the “pulse wave starting point” of one beat.

In step S115, the CPU 11 extracts a predetermined characteristic point from the pulse wave waveform of one beat cut out in step S113, and calculates the TRD in step S117. The sensor signal analyzing process is then terminated.

As described above, the characteristic point necessary for calculating the TRD includes a starting point and an ending point of at least one zone of the vibration zone and the stable zone, and specifically, the pulse wave analyzer according to one or more embodiments of the present invention extracts the starting point and the ending point of the vibration zone in step S115, that is, the convergence time of the reflection wave component of the pulse wave waveform of one beat.

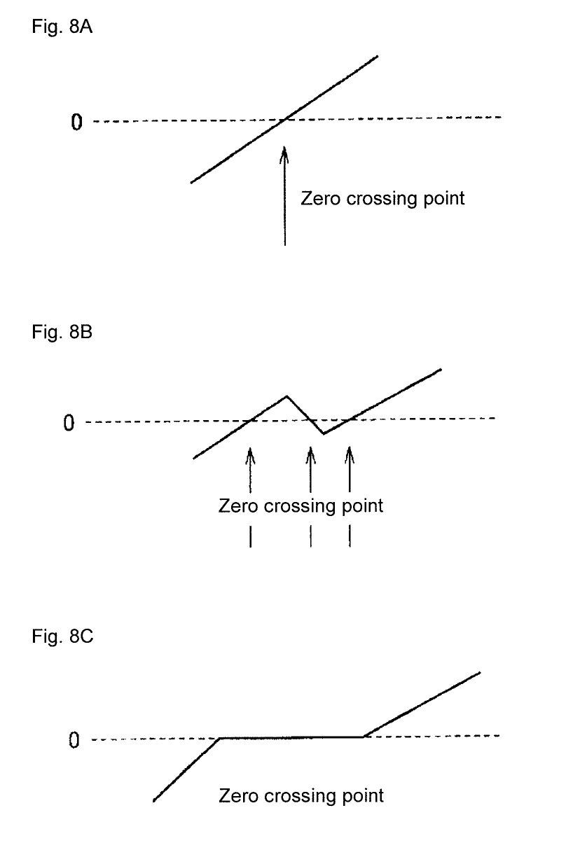

A zero crossing point of the fourth order differentiated wave obtained from the original waveform is often used in the extraction of a general characteristic point. However, for the zero crossing point, a clear zero crossing point may not be extracted as shown in FIG. 8A due to the influence of fluctuation or the like of the base line. As shown in FIGS. 8B and 8C, the zero crossing point may become unclear. FIG. 8B is a case in which the zero crossing point exists in plurals and the zero crossing point to be extracted as the characteristic point of the pulse wave waveform is unclear. FIG. 8C is a case in which the zero crossing point is unclear because a time of zero continues. In the case of the unclear zero crossing point as shown in FIGS. 8B and 8C, the zero crossing point for extracting the characteristic point of the pulse wave may need to be selected. Therefore, the stability lacks if the characteristic point is extracted using the zero crossing point in order to automatically analyze the pulse wave. The stability is required to automatically analyze the pulse wave. Consideration is made in using the fact of not being influenced by fluctuation or the like of the base line such as an extreme point to obtain the stability. The extreme point includes a local maximum point and a local minimum point.

On the premise of representing all signals with Fourier series, the fourth order differentiation of a certain waveform is effective in extracting the high frequency component contained in the relevant signal.

$$\begin{matrix}f ( t ) = sin ( t ) + sin ( 2 t ) \\ \frac{d }{d t} f ( t ) = cos ( t ) + 2 cos ( 2 t ) \\ \frac{{d}^{2}}{d {t}^{2}} = f ( t ) = - sin ( t ) - 4 sin ( 2 t ) \\ \frac{{d}^{3}}{d {t}^{3 }} f ( t ) = - cos ( t ) - 8 cos ( 2 t ) & ( 1 ) \\ \frac{{d}^{4}}{d {t}^{4}} f ( t ) = sin ( t ) + 16 sin ( 2 t ) & ( 2 )\end{matrix}$$

When “sin(2t)” of Equation (1) is fourth order differentiated, it is expressed with “16 sin(2t)” as shown in Equation (2). Therefore, the fourth order differentiation of a certain waveform is found to be effective when extracting the high frequency component contained in the relevant signal.

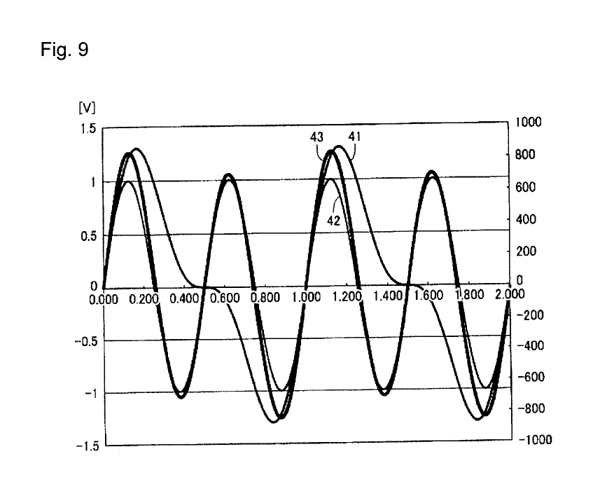

With reference to FIG. 9, a waveform 41 is a waveform representing Equation (1), a waveform 42 is a waveform representing “sin(2t)” in Equation (1), and a waveform 43 is a waveform representing Equation (2). The waveform 43 shows the phase substantially the same as the waveform 42. Therefore, the local maximum point of the high frequency component contained in the signal can be captured as the local maximum point of the fourth order differentiation.

The travelling wave and the reflection wave have high frequency with respect to the pulse wave cycle. Therefore, the maximum point of the travelling wave and the reflection wave is assumed to be extracted by calculating the local maximum point of the fourth order differentiated wave of the pulse wave. The first local maximum point from the rise of the fourth order differentiated wave of the pulse wave waveform of one beat is extracted as the maximum point of the travelling wave, and the next local maximum point can be extracted as the maximum point of the reflection wave. The pulse wave analyzer according to one or more embodiments of the present invention extracts the former local maximum point as the characteristic point indicating the starting point of the vibration zone.

The ending point of the vibration zone is obtained as a converging point of the vibration. Specifically, it is defined as a point where the amplitude of the reflection wave component of the original waveform reaches a defined proportion of the amplitude of the first local maximum point from the rise of the fourth order differentiated wave of the pulse wave waveform of one beat corresponding to the peak of the travelling wave component of the original waveform. The defined proportion is about 10%. The pulse wave analyzer according to one or more embodiments of the present invention extracts the above point as the characteristic point indicating the ending point of the vibration zone.

However, the fourth order differentiated wave easily reacts to even noise of high frequency. Therefore, the maximum point of the travelling wave and the reflection wave serving as the characteristic point of the pulse wave analysis may become difficult to extract.

Equation (3) shows a discrete differentiation formula.

$${f}^{′} ( k ) = \frac{f ( k + 1 ) - f ( k - 1 )}{Δ h} \qquad ( 3 )$$

In the differentiation formula shown in Equation (3), the contained maximum frequency can be adjusted by changing Δh (hereinafter simply referred to as “Δh”) that is an interval taking the difference of data.

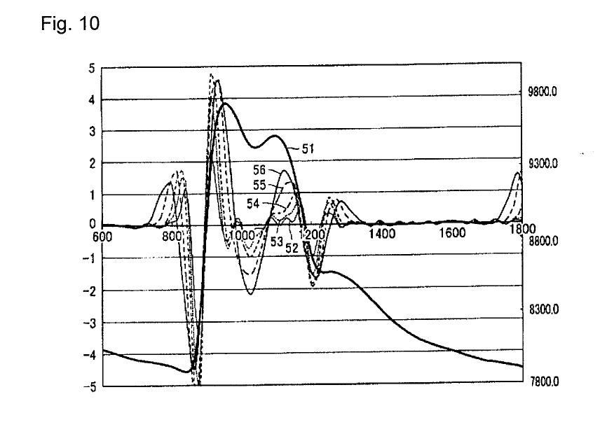

FIG. 10 shows an example in which Δh is 8 ms, 12 ms, 16 ms, 24 ms, and 32 ms with respect to the original waveform. In FIG. 10, the waveform when the value of Δh in fourth order differentiating the original waveform 51 is 8 ms is shown with waveform 52, the waveform when the value of Δh is 12 ms is shown with waveform 53, the waveform when the value of Δh is 16 ms is shown with waveform 54, the waveform when the value of Δh is 24 ms is shown with waveform 55, and the waveform when the value of Δh is 32 ms is shown with waveform 56. With reference to FIG. 10, comparing the waveform 52 and the waveform 56, the amplitude of the waveform 52 is narrower and the component of high frequency is extracted.

The waveform 56 has a gradual amplitude, and only the component of low frequency is extracted. Therefore, the pulse wave component can be selectively extracted by adjusting the frequency characteristics of the fourth order differentiation filter. An actual simulation showed that the characteristic point of the pulse wave can be accurately extracted using the local maximum point of the fourth order differentiation obtained using the fourth order differentiation filter. The result is disclosed in Japanese Laid-Open Patent Publication No. 2005-349116.

The pulse wave analyzer according to one or more embodiments of the present invention extracts the characteristic point of the pulse wave using the extreme point of the fourth order differentiated wave obtained by the fourth order differentiation filter. In the pulse wave analyzer according to one or more embodiments of the present invention, the stability can be enhanced because the zero crossing point of the fourth order differentiation does not need to be used. According to one or more embodiments of the present invention, Δh is set to be longer than the sampling period (2 ms) of the data in the fourth order differentiation filter. Therefore, the noise contained in the high frequency component can be reduced. According to one or more embodiments of the present invention, Δh is assumed as 32 ms.

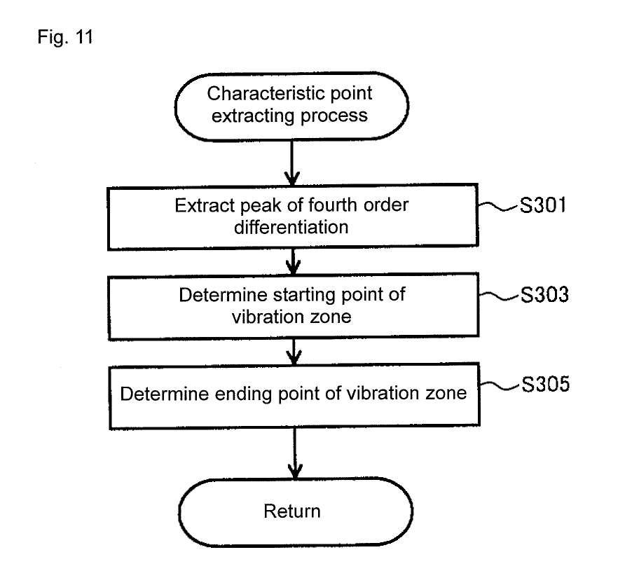

FIG. 11 is a flowchart showing a specific flow of a process for extracting the characteristic point in step S115. With reference to FIG. 11, the CPU 11 obtains the local maximum value of the secondary differentiation existing between PA point and PB point shown in FIG. 7 when recognizing the pulse wave of one beat in step S113. The local maximum value of the secondary differentiation obtained herein is assumed as A point (hereinafter referred to as “APG-A point”), C point (hereinafter referred to as “APG-C point”), and E point (hereinafter referred to as “APG-E point”) in order. In step S301, the CPU 111 acquires the local maximum point of the fourth order differentiation existing from the PA point to the APG-E point. The acquired local maximum point of the fourth order differentiation becomes the candidate of the maximum point of the travelling wave and the reflection wave.

In step S303, the CPU 11 acquires the maximum point of the local maximum point of the fourth order differentiation existing in a zone of a descending limb from the PP point to the APG-E point as the maximum point (P2 point) of the reflection wave, which is one of the characteristic points, and determines such point as the starting point of the vibration zone. The PP point may be a maximum point of the travelling wave or may be a maximum point of the reflection wave. Therefore, the “zone of the descending limb” is merely a zone from the pulse wave maximum point (PP point) to an incisure point (APG-E point). The APG-E point is a point used in analysis as a point representing the timing to close the aorta. Such point on the pulse wave that represents the timing to close the aorta is defined as an “incisure point”. The CPU 11 may also calculate a reflection wave maximum point (P2 point) using the maximum point of the fourth order differentiated wave in the zone from the APG-C point to the APG-E point.

In step S305, the CPU 11 calculates 10% of the amplitude of the PP point serving as the peak of the travelling wave corresponding to the first local maximum point from the rise serving as the PA point shown in FIG. 7 of the fourth order differentiated wave as the threshold value, acquires the zero crossing point of the fourth order differentiated wave after the point at which the amplitude reached the threshold value after the PP point as a converging point of the vibration, which is one of the characteristic points, and determines such point as the ending point of the vibration zone.

After the two characteristic points, the starting point and the ending point of the vibration zone, which are extracted through the above processes, the CPU 11 calculates the TRD that becomes the index by subtracting the time indicating the starting point from the time indicating the ending point in step S117.

The pulse wave analyzer according to one or more embodiments of the present invention extracts the starting point and the ending point of the vibration zone that are easy to extract from the measured pulse wave waveform as characteristic points, and calculates the TR as an index based thereon. As previously described using FIGS. 2 to 5, the TR has a correlation with an index assumed to be useful for diagnosing known heart diseases, and the TR itself is assumed as a useful index. Thus, in the pulse wave analyzer according to one or more embodiments of the present invention, the characteristic point can be extracted from the accurately measured waveform, and the index useful in the diagnosis of the heart disease can be calculated. Not limited to a specific measurement site, the pulse wave can be measured even at the upper arm, and hence, measurements can be easily made at home. Furthermore, because the measurement in the lying position is unnecessary for the measurement body position when measuring the pulse wave at the upper arm, the burden on the person to be measured can be suppressed.

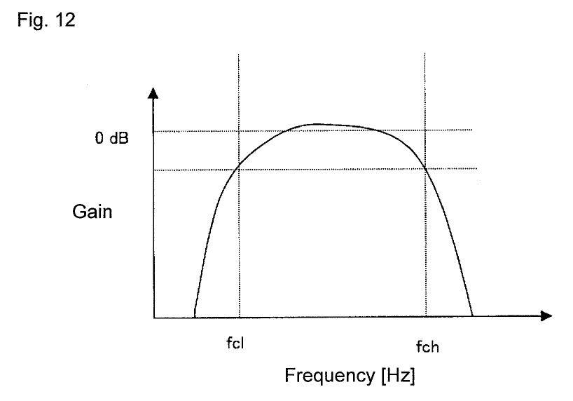

FIG. 12 shows a specific example of a band pass filter used in the digital filtering process of step S109. If the band pass filter shown in FIG. 12 is used for the digital filtering process of step S109, the component having a frequency being smaller than or equal to a value fc1 and the component having a frequency being greater than or equal to fch of the pressure signal digitized in step S107 are removed. In the digital filtering process, the band pass filter is normally used to remove the influence of body motion, so that the frequency lower than a predetermined frequency is removed. The predetermined frequency aimed to remove the influence of body motion is about 0.5 Hz, and 0.5 Hz etc. is set for the threshold value fc1 on the low pass side. It is known from the document “Regional pulse-wave velocity in the arterial tree,” (J Appl Physiol., 1968; January; 24(1):pp. 73-78) by McDonald DA that the pulse wave component having a frequency smaller than 3 Hz may become a factor of error as the pulse wave having a frequency of smaller than 3 Hz differs from the pulse wave having other frequencies in the pulse wave propagation speed. Furthermore, it is known from the document “Estimation of Central Aortic Pressure Waveform by Mathematical Transformation of Radial Tonometry Pressure: Validation of Generalized Transfer Function” (Circulation Vol. 95, No. 7, Apr. 1, 1997, pp. 1827-1836) by Chen-Huan Chen et al. that the pulse wave component having a frequency of smaller than 5 Hz has the amplitude amplified at the stage of propagating to the upper arm when the measurement site is the upper arm. Therefore, according to one or more embodiments of the present invention, 5 Hz is determined for the threshold value fc1 on the low pass side in view of the noise components to remove the body motion, the dependence on the frequency of the propagation speed, and the influence on the pulse wave of each element of amplification of the amplitude at the propagation stage to the upper arm in the digital filtering process of step S109.

In the above example, the fourth order differentiated wave is used to extract the characteristic point from the pulse wave in the pulse wave analyzer, but the band pass filter may also be used in the manner described above. Embodiments of the present invention are not limited to the fourth order differentiated wave as long as the wave is a multi-order differentiated wave of third or greater orders. However, according to one or more embodiments of the present invention, the fourth order differentiated wave is used because the accuracy for obtaining the characteristic point is experimentally high in the fourth order differentiated wave.

The process of extracting the starting point and the ending point of the vibration zone as the characteristic point in step S115 is not limited to the above method. In other words, another method of such process includes a method of calculating the moving average value of the fourth order differentiated wave of the pulse wave of one beat, extracting the point at which the maximum value is reached is extracted as the starting point of the vibration zone, and extracting the point at which the moving average value does not exceed a value smaller by the defined proportion from the maximum value after reaching the maximum value as the ending point of the vibration zone.

In the above description, a configuration of detecting the pulse wave by capturing the change in pressure using the pressure sensor is adopted, but the method of detecting the pulse wave is not limited to such configuration. For instance, a method of detecting the pulse wave by capturing the change in volume may be adopted.

The method of analyzing the pulse wave waveform described above is not limited to the analysis of the pulse wave waveform, and may be used to analyze other biological waves obtained by synthesizing a first waveform and a second waveform generated by contraction and expansion of the heart such as the heart beat waveform. Furthermore, the analysis of the pulse wave in the pulse wave analyzer, that is, the method of extracting the characteristic point and the method of calculating the index may be provided as a program. Such program may be recorded in a computer readable recording medium such as a flexible disc, a CD-ROM (Compact Disk-Read Only Memory), a ROM (Read Only Memory), RAM (Random Access Memory), a memory card or the like adjunct to the computer, and provided as a program product. Alternatively, the program may be provided by being recorded in a recording medium such as a hard disc incorporated in the computer. The program may also be provided by being downloaded through a network.

The program according to one or more embodiments of the present invention may be for calling out the necessary module at a predetermined timing in a predetermined array and executing the process of the program modules provided as one part of the operating system (OS) of the computer. In this case, the relevant module is not included in the program itself and is operated cooperatively with the OS to execute the process. The program according to one or more embodiments of the present invention also includes the program that does not include such module.

The program according to one or more embodiments of the present invention may be provided by being incorporated in one part of another program. In this case as well, the module included in the other program is not included in the program itself and is operated cooperatively with the other program to execute the process. The program according to one or more embodiments of the present invention also includes the program incorporated in the other program.

The program product to be provided is installed in a program storage unit such as a hard disc, and executed. The program product includes the program itself and the storage medium in which the program is recorded.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1 sensor unit
3 display unit
5 air tube
7 fixing stand
11 CPU
12 ROM
13 RAM
14 control circuit
15 pressurization pump
16 negative pressure pump
17 switching valve
18 pushing cuff
19 semiconductor pressure sensor
20 multiplexer
21 amplifier
22 characteristic variable filter
23 A/D converter
24 operating section
25 display section

1. A pulse wave analyzer comprising:
a pulse wave detection unit that detects a pulse wave; and
a calculation device that carries out a process based on the pulse wave detected by the pulse wave detection unit,
wherein the process carried out by the calculation device comprises:
a process of extracting a characteristic point for sectionalizing a reflection wave zone from a pulse wave waveform of one beat; and
a process of calculating a convergence time of the reflection wave as an index.

2. The pulse wave analyzer according to claim 1, further comprising:
a digital conversion unit that converts the pulse wave signal from the pulse wave detection unit into a digital signal; and
a fourth order differentiation filter enabling adjustment of frequency characteristics for obtaining a fourth order differentiated wave of an original waveform based on the digital signal converted by the digital conversion unit,
wherein the process carried out by the calculation device further comprises a process of calculating an extreme point of the fourth order differentiated wave in a zone of a pulse wave of one beat, and
wherein the process of extracting the characteristic point comprises:
a process of extracting a starting point of the reflection wave zone based on the extreme point of the fourth order differentiated wave, and
a process of extracting an ending point of the reflection wave zone based on an amplitude of the fourth order differentiated wave.

3. The pulse wave analyzer according to claim 2,
wherein in the process of extracting the starting point of the reflection wave zone, a local maximum point of the first fourth order differentiated wave from a rising point of the pulse wave of a first beat is extracted as the characteristic point that is the starting point of the reflection wave zone, and
wherein in the process of extracting the ending point of the reflection wave zone, a point where an amplitude of the pulse wave reached a defined proportion after a point corresponding to an extreme point is extracted from the amplitude of the pulse wave of a point corresponding to the extreme point of the first fourth order differentiated wave from the rising point of the pulse wave of one beat as the characteristic point or the ending point of the reflection wave zone.

4. The pulse wave analyzer according to claim 2,
wherein in the process of extracting the starting point of the reflection wave zone, a point where a moving average value of the fourth order differentiated wave of one beat is a maximum is extracted as the characteristic point or the starting point of the reflection wave zone, and
wherein in the process of extracting the ending point of the reflection wave zone, a point where the moving average value does not exceed a value smaller by a defined proportion from the maximum value after reaching the point where the moving average value of the fourth order differentiated wave of one beat is the maximum is extracted as the characteristic point that is the ending point of the reflection wave zone.

5. The pulse wave analyzer according to claim 2, wherein the process carried out by the calculation device further comprises a filtering process for offsetting and excluding a noise component by a moving average value of the fourth order differentiated wave in a zone of the pulse wave of one beat.

6. A pulse wave analyzing method comprising the steps of:
extracting a characteristic point for sectionalizing a reflection wave zone from a pulse wave waveform of one beat obtained with a pressure sensor for detecting a pulse wave; and
calculating a convergence time of a reflection wave as an index.

7. A program for causing a computer to execute a process of analyzing a pulse wave and calculating an index, the program causing the computer to execute the steps of:
acquiring a sensor signal from a pressure sensor for detecting a pulse wave;
extracting a characteristic point for sectionalizing a reflection wave zone from a pulse wave waveform of one beat based on the sensor signal; and
calculating a convergence time of a reflection wave as an index.
